(19) United States
(12) Patent Application Publication
(10) Pub. No.: US 2009/0148538 A1
(43) Pub. Date: Jun. 11, 2009

(54) USE OF ADSORBENT CARBON MICROSPHERES TO TREAT POUCHITIS

(75) Inventors: Laurent Fischer, Del Mar, CA (US); Matthew Scott Harris, Leesburg, VA (US)

Correspondence Address: KNOBBE MARTENS OLSON & BEAR LLP, 2040 MAIN STREET, FOURTEENTH FLOOR, IRVINE, CA, 92614, US

(73) Assignee: Ocera Therapeutics, Inc., San Diego, CA (US)

(21) Appl. No.: 12/249,853

(22) Filed: Oct. 10, 2008

Related U.S. Application Data
(60) Provisional application No. 60/979,495, filed on Oct. 12, 2007.

(51) Int. Cl. A61K 33/44; A61P 1/00

(52) U.S. Cl. 424/600

(57) ABSTRACT

Disclosed herein is the use of adsorbent carbon microspheres for the treatment of pouchitis.

### RELATED APPLICATION

This application claims the benefit of U.S. Provisional Application No. 60/979,495, filed Oct. 12, 2007, which is incorporated herein by reference in its entirety.

### BACKGROUND

1. Field of the Invention

One aspect of the present invention relates to the treatment of pouchitis and symptoms associated with pouchitis using adsorbent carbon.

2. Description of the Related Art

Ulcerative colitis affects at least a half million Americans and incidence appears to be increasing. In the US, the reported incidence of ulcerative colitis is approximately 8 per 100,000 person year and the prevalence is about 200 per 100,000 population. Approximately 30% of patients with ulcerative colitis eventually require proctocolectomy. Ileal pouch-anal anastomosis (IPAA) with proctocolectomy has become the surgical treatment of choice for patients with ulcerative colitis who fail medical therapy or develop dysplasia. After the proctocolectomy is performed, the remaining ileum is made into a reservoir (“pouch”) and connected to the anus. The pouch may be formed in different configurations (J-pouch, S-pouch, and others). The IPAA avoids a permanent ileostomy and permits stool to be retained and eliminated as a bowel movement. The operation may be performed in multiple stages with an interim temporary ileostomy. While IPAA with proctocolectomy significantly decreases the risk for dysplasia or cancer, reduces the need for long-term medicines, and improves patients' health-related quality of life, short-term and long-term complications can occur including pouchitis.

### SUMMARY

One embodiment disclosed herein includes a method of treating one or more symptoms of pouchitis, comprising administering to a subject suffering from said symptoms adsorbent carbon. In some embodiments, the adsorbent carbon is adsorbent carbon microspheres having a particle size of about 0.01 to about 2 mm.

Another embodiment disclosed herein includes a method of treating one or more symptoms of pouchitis in a patient who continues to be symptomatic after administration of an antibiotic, the method comprising administering to the patient adsorbent carbon microspheres having a particle size of about 0.01 to about 2 mm.

Another embodiment disclosed herein includes a method of treating one or more symptoms of pouchitis, comprising co-administering to a subject an antibiotic and adsorbent carbon microspheres having a particle size of about 0.01 to about 2 mm.

Another embodiment disclosed herein includes a method of treating one or more symptoms of pouchitis in a patient who is antibiotic dependent including administering to the patient adsorbent carbon microspheres having a particle size of about 0.01 to about 2 mm.

Another embodiment disclosed herein includes a method of treating one or more symptoms of pouchitis including co-administering to a subject a probiotic and adsorbent carbon microspheres having a particle size of about 0.01 to about 2 mm.

Another embodiment disclosed herein includes a method of treating one or more symptoms of pouchitis including co-administering to a subject a TNF alpha inhibitor and adsorbent carbon microspheres having a particle size of about 0.01 to about 2 mm.

### BRIEF DESCRIPTION OF THE DRAWING

FIG. 1 is a series of bar graphs depicting the change in Pouchitis Disease Activity Index (PDAI) scores upon administration of AST-120.

### DETAILED DESCRIPTION

### Definitions

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional such as a Medical Doctor (i.e. Doctor of Allopathic medicine or Doctor of Osteopathic medicine) or a Doctor of Veterinary Medicine to attempt to cure, or at least ameliorate the effects of, a particular disease or disorder or to prevent the disease or disorder from occurring in the first place.

As used herein, a “dosage” refers to an amount of therapeutic agent administered to a patient.

As used herein, a “daily dosage” refers to the total amount of therapeutic agent administered to a patient in a day.

As used herein, the term meq means milliequivalents(s).

As used herein, the term “therapeutic agent” means a substance that is effective in the treatment of a disease or condition. For example, the therapeutic agent(s) may include antibiotics or probiotics.

### Treatment of Pouchitis

Pouchitis is the inflammation of the ileal pouch. Pouchitis is the most common complication of IPAA with a reported annual incidence of 40% and a cumulative frequency of up to 50% during a 10 year follow-up period. Patients with pouchitis typically present with diarrhea, abdominal pain, and pelvic discomfort. Endoscopy in patients with pouchitis usually reveals erythematous pouch mucosa, loss of pseudocolonic vasculature or other architecture, and friability of the mucosa. Biopsies show evidence of inflammatory cells or red blood cells in the lamina propria. Pouchitis is usually treated with antibiotics, and specifically usually with ciprofloxacin and/or metronidazole. Probiotics such as VSL-3 and Bio-K+ may be useful as prophylactic agents that spare antibiotic therapy.

The Pouchitis Disease Activity Index (PDAI) is the most commonly used instrument for grading the severity of pouchitis (described in Sandborn W J, Tremaine W J, Batts K P, Pemberton J H, Phillips S F. Pouchitis after ileal pouch-anal anastomosis: A pouchitis disease activity index. Mayo Clin Proc 1994; 69:409-15, which is incorporated herein by reference in its entirety). The PDAI applies quantitative scores to clinical symptoms and endoscopic and histologic acute inflammation. Pouchitis has been defined as a total of PDAI score=7 points. From various perspectives, pouchitis may be categorized into: 1) remission vs. active; 2) acute vs. chronic; and 3) infrequent episodes vs. relapsing vs. continuous course. Another classification is based on the response to antibiotic therapy. Analogous to the classification of ulcerative colitis according to the response or dependency to corticosteroids, pouchitis can be classified based on the manner of patient's response to antibiotics: antibiotic-responsive pouchitis, antibiotic-dependent pouchitis, and antibiotic-refractory pouchitis.

Although most patients with active pouchitis respond favorably to antibiotic therapy, relapse is common, which often requires frequent or continuous therapy. Concerns regarding frequent use of antibiotic agents include: 1) antibiotics often have adverse effects, including direct side-effects or colonization with Clostridium difficile; 2) long-term or frequent use of antibiotics, including ciprofloxacin, metronidazole, and rifaximin, may lead to bacterial resistance. More and more patients with antibiotic-refractory pouchitis are appearing, which could largely be due to overuse of antibiotic agents; 3) ulcerative colitis patients with IPAA have an increased risk for the development of intra-abdominal infections, such as pouch leaks, abscess, and cholangitis from primary sclerosing cholangitis, which require antibiotic therapy with the agents similar to the agents used in pouchitis. Bacterial resistance developed from the overuse of antibiotics for pouchitis might jeopardize the treatment of other intra-abdominal infections; 4) overuse of antibiotics can lead to overgrowth of various commensal or pathogenic bacteria, resulting in pouch inflammation.

In some embodiments, pouchitis or symptoms associated with pouchitis are treated or ameliorated by administering a therapeutic amount of an adsorbent carbon to a patient. In some embodiments, the adsorbent carbon is activated carbon. In some embodiments, the adsorbent carbon is adsorbent carbon microspheres. As used herein, an “adsorbent carbon microsphere” is a particle having a spherical or spheroid-like shape whose composition is mostly carbon and which has adsorbent properties. In some embodiments, the carbon microspheres have diameters from about 0.01 to about 2 mm. In some embodiments, the diameters are from about 0.02 to about 1 mm. In still other embodiments, the diameters are from about 0.05 to about 0.8 mm. In a typical embodiment, the carbon microspheres can have diameters from about 0.1 to about 0.5 mm. For example, the carbon microspheres can have diameters from about 0.2 to about 0.4 mm.

In some embodiments, the adsorbent carbon microspheres have a specific surface area of about 700 m²/g or more, such as determined by a BET (Brunauer-Emmett-Teller theory model) method (Brunauer et al. “Adsorption of Gases in Multimolecular Layers”, J. Am. Chem. Soc., 1938, 60(2), 309-319, which is incorporated herein by reference in its entirety). In some embodiments, the specific surface area is from about 700 m²/g to about 2500 m²/g. In a typical embodiment, the specific surface area is from about 1400 m²/g to about 1900 m²/g. For example, the specific surface area may be from about 1500 m²/g to about 1800 m²/g. In one embodiment, the specific surface area is greater than about 1600 m²/g. In some embodiments, the volume of pores in the carbon microspheres having a pore diameter of about 20 to about 15,000 nm are from about 0.04 mL/g to about 0.10 mL/g.

In some embodiments, the total amount of acidic groups on the carbon are from about 0.30 to about 1.20 meq/g. In a typical embodiment, the total amount of acidic groups on the carbon is from about 0.30 to about 0.80 meq/g. For example, the total amount of acidic groups on the carbon may be from about 0.40 to about 0.70 meq/g. In some embodiments, the total amount of basic groups on the carbon are from about 0.20 to about 1.00 meq/g. In a typical embodiment, the total amount of acidic groups on the carbon can be from about 0.30 to about 0.80 meq/g. For example, the total amount of acidic groups on the carbon may be from about 0.35 to about 0.65 meq/g.

Suitable forms of adsorbent carbon microspheres are also described in U.S. Pat. Nos. 4,681,764 and 6,830,753 and U.S. Application Publication Nos. 2005/0112114; 2005/0079167; and 2005/0152890; all of which are incorporated herein by reference in their entirety.

In one embodiment, the adsorbent carbon microspheres are AST-120, available under the trade name KREMEZIN® from Kureha Corp. (Japan). AST-120 is a spherical activated carbon produced from pitch, such as by the process disclosed in U.S. Pat. No. 4,681,764. AST-120 has a particle size of about 0.2 to about 0.4 mm and is a homogeneous spherical particle (not a spherical particle produced by granulating a carbon powder).

In some embodiments, administration of adsorbent carbon microspheres to a patient suffering from pouchitis results in a decrease of one or more pouchitis-related symptoms, including but not limited to, diarrhea, abdominal pain, pelvic discomfort, bloody diarrhea, urgency in passing stools, or discomfort while passing stools, or an improvement of endoscopically visualized abnormalities of the pouch, including erythematous pouch mucosa, loss of pseudocolonic vasculature or other architecture, friability of the mucosa, and presence of inflammatory cells or red blood cells in the lamina propria. In some embodiments, administration of adsorbent carbon microspheres to a patient suffering from pouchitis results in an improvement in the Pouchitis Disease Activity Index (PDAI). In one embodiment, the PDAI at least about a 3 point reduction in the PDAI is achieved. In some embodiments, administration of adsorbent carbon microspheres to a patient suffering from pouchitis results in a reduction of the PDAI clinical symptom score, a reduction of the PDAI endoscopic score, and/or a reduction of PDAI histology score. In one embodiment, a PDAI score <7 is achieved, signifying disease remission. In some embodiments, administration of adsorbent carbon microspheres to a patient suffering from pouchitis results in an improvement in quality of life as evidenced by the Cleveland Global Quality of Life (CGQL) questionnaire (as described in Fazio V W, O'Riordain M G, Layery I C, Church J M, Lau P, Strong S A, Hull T., Long-term functional outcome and quality of life after stapled restorative proctocolectomy. Ann Surg. 1999 October; 230(4):575-84, which is incorporated herein by reference in its entirety) and/or the Short Inflammatory Bowel Disease Questionnaires (SIBDQ) (as described in Irvine E J, Zhou Q, Thompson A K, The Short Inflammatory Bowel Disease Questionnaire: a quality of life instrument for community physicians managing inflammatory bowel disease. Am J Gastroenterol. 1996 August; 91(8):1571-8, which is incorporated herein by reference in its entirety).

In some embodiments, adsorbent carbon microspheres such as AST-120 are administered in combination with one or more therapeutic agent(s) to treat pouchitis. In some embodiments, the one or more therapeutic agent(s) include one or more antibiotics, including ciprofloxacin, metronidazole, or rifaximin. In some embodiments, the one or more therapeutic agent(s) include one or more probiotics, including VSL-3 and Bio-K+. In some embodiments, adsorbent carbon microspheres such as AST-120 are used to treat pouchitis in patients who are antibiotic-refractory, and who continue to be symptomatic after treatment with an antibiotic such as ciprofloxacin, metronidazole, or rifaximin and/or a probiotic such as VSL-3 and Bio-K+. In some embodiments, adsorbent carbon microspheres such as AST-120 are used to treat pouchitis in patients who are antibiotic-dependent, and who rely on continuous treatment with an antibiotic such as ciprofloxacin, metronidazole, or rifaximin and/or a probiotic such as VSL-3 and Bio-K+. In some embodiments, the one or more therapeutic agent(s) include a TNF alpha inhibitor, including but not limited to infliximab, adalimumab, or certolizumab. In some embodiments, the one or more therapeutic agent(s) include a corticosteroid, including but not limited to dexamethasone, prednisone, fludrocortisone, and hydrocortisone. These corticosteroids may be administered orally or by enema. In other embodiments, the one or more therapeutic agent(s) include mesalamine. The mesalamine can likewise be administered orally or by enema. In some embodiments, the one or more therapeutic agents may include an immodulatory agent, including but not limited to azathioprine, 6-mercaptopurine, cyclosporine, or tacrolimus.

In some embodiments, the adsorbent carbon microspheres are administered orally. In other embodiments, the adsorbent carbon microspheres are administered rectally. For example, in one embodiment, the adsorbent carbon microspheres are administered via enema directly to the IPAA. The dosage of the adsorbent carbon microspheres, either administered alone or in combination with another therapeutic agent, may be from about 1 to 20 grams per day. In some embodiments, the daily dosage of the adsorbent carbon microspheres may be divided into multiple administrations (e.g. into two to four portions daily). In some embodiments each portion can be from about 1 g to about 6 g (e.g., about 2 g to about 3 g). In a typical embodiment, a patient takes from about 2 g to about 3 g of adsorbent carbon microspheres (e.g., AST-120) orally three times daily. In some embodiments, each portion may be taken as often as four times daily.

In some embodiments, the adsorbent carbon microspheres can be prescribed or administered at a specific dosage per day. In other embodiments, the patient can be instructed to take the composition when he or she experiences one or more symptoms related to a condition being treated. For example, the patient may be instructed to take a composition when he is experiencing severe diarrhea.

As used herein, by administration in “combination,” it is meant that the adsorbent carbon microspheres are in the patient at the same time as one or more therapeutic agents may be found in the patient's bloodstream or stomach, regardless of when or how the adsorbent carbon microspheres and therapeutic agents are actually administered. In one embodiment, the adsorbent carbon microspheres and the therapeutic agent(s) are administered simultaneously. In one such embodiment, administration in combination is accomplished by combining the adsorbent carbon microspheres and the therapeutic agent(s) in a single dosage form. In another embodiment, the adsorbent carbon microspheres and the therapeutic agent(s) are administered sequentially. In some embodiments, the adsorbent carbon microspheres and the therapeutic agent(s) (e.g., antibiotics or probiotics) are administered about 20 minutes to about 1 hour apart from each other (e.g., about 30 minutes apart). In one embodiment the adsorbent carbon microspheres and the therapeutic agent(s) are administered through the same route, such as orally. In another embodiment, the adsorbent carbon microspheres and the therapeutic agent(s) are administered through different routes, such as one being administered orally and another being administered i.v.

### Production of Adsorbent Carbon Microspheres

Adsorbent carbon microspheres suitable for use as described herein may be produced by any suitable method, including but not limited to the following:

First, a bicyclic or tricyclic aromatic compound or a mixture thereof having a boiling point of 200° C. or more is added as an additive to a pitch such as a petroleum pitch or a coal pitch. The whole is heated and mixed, and then shaped to obtain a shaped pitch. Thereafter, the shaped pitch is dispersed and granulated in hot water at 70 to 180° C., with stirring, to obtain a microspherical shaped pitch. The aromatic additive is extracted and removed from the shaped pitch by a solvent having a low solubility to the pitch but a high solubility to the additive. The resulting porous pitch is oxidized by an oxidizing agent to obtain a porous pitch subject to heat infusibility. The resulting infusible porous pitch is treated at 800 to 1000° C. in a gas flow such as steam or carbon dioxide gas reactive with carbon to obtain a porous carbonaceous substance.

The resulting porous carbonaceous substance is then oxidized in a temperature range of 300 to 800° C., preferably 320 to 600° C., in an atmosphere containing 0.1 to 50% by volume, preferably 1 to 30% by volume, particularly preferably 3 to 20% by volume, of oxygen. The substance is thereafter reduced in a temperature range of 800 to 1200° C., preferably 800 to 1000° C., in an atmosphere of a non-oxidizable gas to obtain the final product.

In one alternative, the adsorbent carbon microspheres may be produced from a resin instead of a pitch. More details of suitable production processes and suitable products may be found in U.S. Pat. Nos. 4,681,764; 6,830,753; and U.S. Application Publication No. 2005/0112114, filed May 26, 2005, all of which are incorporated herein by reference in their entirety. Suitable adsorbent carbon microspheres are commercially available from Kureha Corp., and are sold in Japan under the trade name KREMEZIN® (also known as AST-120).

### Pharmaceutical Compositions

For use as described herein, adsorbent carbon microspheres may be administered to the gut of a subject by any suitable means. In one embodiment, the adsorbent carbon microspheres are administered orally. Formulations for oral administration may include, but are not limited to, the free flowing microspheres, granules, tablets, sugar-coated tablets, capsules, suspensions, sticks, divided packages, or emulsions. In the case of capsules, gelatin capsules, or if necessary, enteric capsules may be used. In the case of tablets, the formulations may advantageously be adapted to break into the original fine particles inside the body. In the case of free flowing microspheres, the formulations may be in a sachet that is opened immediately prior to ingestion. The adsorbent may be used as a mixture with an electrolyte-controlling agent, such as an aluminum gel or KAYEXALATE® (Winthrop Lab, U.S.A.) or other agents. The microspheres may be ingested with the aid of a liquid or soft food (e.g., apple sauce). In another embodiment, the adsorbent carbon microspheres are administered rectally.

Certain preparations for oral use can be obtained by mixing one or more excipients with adsorbent carbon microspheres as described herein and processing the mixture after adding suitable auxiliaries, if desired, to obtain tablets or dragee cores. The term “carrier” material or “excipient” herein can mean any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration.

Excipients can include, by way of illustration and not limitation, diluents, disintegrants, binding agents, adhesives, wetting agents, polymers, lubricants, glidants; substances added to mask or counteract a disagreeable texture, taste or odor; flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants.

A variety of techniques for formulation and administration can be found in Remington: The Science and Practice of Pharmacy (20^{th} ed, Lippincott Williams & Wilkens Publishers (2003)), which is incorporated herein by reference in its entirety.

The dosage form used may be any amount of adsorbent carbon microspheres suitable to achieve the desired therapeutic result. In some embodiments, each unit dose is from about 1 g to about 6g (e.g., about 2 g to about 3 g). In some embodiments, dosages of the adsorbent are individually packaged so as to preserve the absorptivity of the material. For example, divided packaging may be used such as described in more detail in U.S. Pat. No. 5,686,081, which is incorporated herein by reference in its entirety. The divided packaging may contain unit doses of the adsorbent carbon microspheres in their free flowing form.

### EXAMPLES

### Example 1

An open-label pilot trial in which patients suffering from pouchitis receive AST-120 in 2g sachets (oral) three times a day for 4 weeks is conducted. All antibiotics are discontinued for at least 2 weeks prior to study entry. Probiotics and nutritional agents are at stable dose or discontinued prior to the study. In an extension cohort, patients who are considered responders (i.e., have had at least a 3 point reduction in the 18 point PDAI scoring system) or who are in remission (PDAI<7) are eligible to receive open-label AST-120 for as long as response is maintained for a minimum of 12 weeks and up to a maximum of 52 weeks. One study population includes antibiotic responders or antibiotic naïve patients. A second study population includes antibiotic refractory patients. For the antibiotic responders or antibiotic naïve patients, the primary efficacy endpoint of the four-week cohort is remission induction as defined by a Pouchitis Disease Activity Index (PDAI) score <7 at four weeks of therapy; and for a second cohort, maintenance of remission over 12 months. The secondary efficacy endpoints include response defined as a=3 point reduction in the 18-point PDAI scoring system, reduction of PDAI clinical symptom score (e.g., stool frequency returns to the normal baseline), reduction of PDAI endoscopic score, and reduction of PDAI histology score. For antibiotic refractory patients, the primary efficacy endpoint is a decrease in the PDAI score of at least 3 points at four weeks of therapy. Secondary efficacy endpoints include remission (PDAI<7), reduction in PDAI clinical score of at least 1 point, reduction in PDAI endoscopic score of at least 1 point, and reduction in PDAI histology score of at least 1 point.

Study Population-Antibiotic Responder or Antibiotic Naïve

Patients included in the antibiotic responder or antibiotic naïve population have active pouchitis after ileal pouch-anal anastomosis for ulcerative colitis present with primary symptoms such as increased stool frequency and abdominal pain. The diagnosis of active pouchitis is defined by a PDAI score >7 points, with a combined assessment of symptoms, endoscopy, and histology. The patients included have never been treated with antibiotics (antibiotic-naïve) or have been previously treated with antibiotics and responded. Antibiotic-dependent patients may be enrolled as long as antibiotic use is discontinued for at least two weeks prior to study entry. Antibiotic-dependent pouchitis is defined as a condition in which a patient with frequent episodes (>4 episodes per year) of pouchitis or persistent symptoms requires long-term, continuous antibiotic therapy to keep the disease in remission.

### Study Population-Antibiotic Refractory

Patients included in the antibiotic refractory population have active pouchitis after ileal pouch-anal anastomosis for ulcerative colitis and do not respond to antibiotic therapy for a minimum of 14 days. The subjects have active pouchitis as confirmed by endoscopy and biopsy within 4 seeks of study entry and have a PDAI score >7 points, with a combined assessment of symptoms, endoscopy, and histology.

### Exclusion Criteria

Patients are excluded who were previously treated with infliximab or any investigational immunosuppressant/immunomodulator; antibiotic responders or antibiotic naïve patients whose condition is severe enough that, in the investigator's opinion, withholding antibiotics for 4 weeks during the AST-120 trial is not feasible; who are undergoing chemotherapy for the treatment of cancer; who used antibiotics within 2 weeks prior to entry in the study; who have Crohn's disease of the pouch, including inflammatory, fibrostenotic, or fistulizing phenotypes, based on the previously established diagnostic criteria; who have active specific infection of the pouch with cytomegalovirus infection or C. difficile infection; for the antibiotic responders or antibiotic naïve population, patients who have chronic antibiotic-refractory pouchitis (defined as a condition where a patient fails to respond to a 4 week course of a single antibiotic (metronidazole or ciprofloxacin), requiring prolonged therapy of >4 weeks consisting of 2 antibiotics, oral or topical 5-aminosalicylate, corticosteroid therapy, or oral immunomodulator therapy) or antibiotic-dependent pouchitis (defined as a condition in which a patient with frequent episodes (>4 episodes per year) of pouchitis or persistent symptoms requires long-term, continuous antibiotic or probiotic therapy to keep the disease in remission); who have a history of non-inflammatory disease of the pouch including decreased pouch compliance, irritable pouch syndrome, and afferent or efferent limb obstruction; antibiotic responders or antibiotic naïve patients who have isolated cuffitis; who have strictures of the pouch inlet or outlet; who have familial adenomatous polyposis; who have a history of lactose intolerance; who have known celiac disease; who have primary sclerosing cholangitis (PSC) with or without liver transplant or with or without Actigall® or Urso® therapy; who have uncontrolled systemic diseases; who need oral or topical steroid treatment or 5-ASA agents; who have other major physical or major psychiatric illness within the last 6 months that in the opinion of the investigator would affect the patient's ability to complete the trial; who actively use cholestyramine; who actively use NSAIDs or aspirin; or who are on therapy with 5-ASA at the entry of the study.

### Treatment Regimen and Duration and Co-Medications

AST-120 sachets at 2g are administered three times daily (TID) taken between meals and immediately before going to bed for 4 complete weeks. Administration is continued for 52 weeks for responders (extension cohort). Any co-prescribed medicines are given at least 30 minutes before AST-120 administration. Permitted co-prescribed medicines include antidiarrheal agents (e.g., Imodium, loperamide) if the dose has been stabilized for 2 weeks at the entry of the trial.

### Study Procedures and Monitoring

Patients are checked by telephone on a weekly basis for symptom response, compliance, and development of AEs. Patients who are considered responders (i.e., have had at least a 3 point reduction in the 18 point PDAI scoring system) or who are in remission (PDAI<7) are eligible to receive open-label AST-120 for as long as response is maintained up to a maximum of 52 weeks. If, at any time the patient relapses (PDAI>7 for patients who were in remission, or an increase in PDAI such that at least a 3 point reduction is not maintained for responders), open label treatment is discontinued and the patient is removed from the study. Patients also discontinue open-label treatment and are removed from the study if warranted by treatment-emergent safety concerns or if, in the opinion of the investigator, it is in the patient's best interests to discontinue the study.

### Study Materials

### Packaging and Labeling

AST-120 is packaged in 2 g quantities in PVC-coated aluminum foil sachets. The sachets are attached in strips of 3 sachets each. Each day's dose is 3 sachets or one strip. The sachets are provided in cardboard secondary containers packaged as kits containing a 4 week supply of product for each patient. Each kit contains an overage (4 additional days supply) to allow for flexibility in scheduling follow-up visits.

### Product Administration

Patients are instructed to take 3 sachets per day between meals at 10:00 a.m., 3:00 p.m., and immediately before going to bed. For each administration, the patient tears open the sachet (along the pre-perforated “scissor-line” demarcation), drops the contents of the sachet directly on their tongue, and swallows the investigational product using as much water as necessary to comfortably “wash down” the product and clear their mouths of any residue (at least 8 ounces).

### Data Analysis

Descriptive statistics are computed for all factors. These include means and standard deviations for continuous factors and frequencies for categorical factors. The clinical remission rate is estimated by calculating the percentage of subjects who have a PDAI<7 at week 4. The clinical response rate is estimated by calculating the percentage of subjects with a reduction in PDAI of 3 points or more. For each proportion the corresponding 95% confidence interval is estimated. In addition, the percentage of subjects who experience adverse effects to the study agent is also estimated. The mean and standard deviation of the change in health-related quality-of-live scores are estimated.

For the subgroup of subjects who respond to 4 weeks of acute treatment, they are contacted weekly by the study coordinator to assess for recurrence of symptoms of pouchitis. This follow up data is reported as number and percent who maintain remission, and time-to-recurrence according to the Kaplan-Meier survival method.

### Results

A total of 20 patients enrolled in the trial and received AST-120. A total of 19 patients were included in the final analysis. One patient developed an upper respiratory infection on the second day, discontinued treatment and was excluded from the final analysis. None of the patients had significant laboratory abnormalities at baseline. All the patients tested negative for Clostridium difficile toxins A or B at entry into the trial.

Demographic and clinical data from the 19 patients enrolled in the trial are listed in Table 1. Concurrent medicines and significant past medical history are also shown in the table.

TABLE 1
Demographic and Clinical Data (N = 19).

| Variable | Overall Summary |
|---|---|
| Age, years | 44.26 ± 11.39 |
| Male gender | 13 (68.4%) |
| Caucasian race | 19 (100%) |
| Family history of Crohn's disease | 1 (5.3%) |
| Family history of ulcerative colitis | 2 (10.5%) |
| Family history of colon cancer | 0 |
| Family history of celiac disease | 1 (5.3%) |
| Current smoker | 2 (10.5%) |
| Ex-smoker | 6 (31.6%) |
| Regular alcohol drinker | 1 (5.3%) |
| Osteoporosis | 2 (10.5%) |
| Renal stones | 4 (21.1%) |
| Depression | 2 (10.5%) |
| Coronary artery disease | 2 (10.5%) |
| Hepatitis C | 1 (5.3%) |
| Breast cancer | 1 (5.3%) |
| Current wt loss >5% | 2 (10.5%) |
| Pre-op diagnosis of ulcerative colitis | 16 (84.2%) |
| Pre-op diagnosis of Indeterminate colitis | 3 (15.8%) |
| Duration of ulcerative/indeterminate colitis, yrs | 15.00 ± 7.86 |
| Pancolitis | 17 (94.4%) |
| Colectomy for refractory colitis | 15 (78.9%) |
| Colectomy for dysplasia | 4 (21.1%) |
| Duration of pouch, yrs | 6.05 ± 4.12 |
| J pouch configuration | 18 (94.7%) |
| 2-stage of pouch surgery | 10 (55.6%) |
| History of Pouchitis | |
| None | 1 (5.3%) |
| Antibiotic-responsive pouchitis | 2 (10.5%) |
| Antibiotic-dependent pouchitis | 13 (68.4%) |
| Antibiotic-refractory pouchitis | 3 (15.8%) |
| Extra-intestinal Manifestations | |
| Arthralgia | 9 (47.4%) |
| Thromboembolic events | 1 (5.3%) |
| Concurrent Meds at the entry of and during trial | |
| None | 5 (26.3%) |
| Antidiarrheal | 7 (36.8%) |
| Oral or topical narcotics | 3 (15.8%) |
| Topical or oral 5-ASAs | 1 (5.3%) |
| Antidepressants | 3 (15.8%) |
| Antianxiety | 2 (10.5%) |
| Topical corticosteroid | 1 (5.3%) |
| Oral budesonide | 1 (5.3%) |
| Aspirin | 2 (10.5%) |
| Statins | 2 (10.5%) |
| Estrogens | 1 (5.3%) |

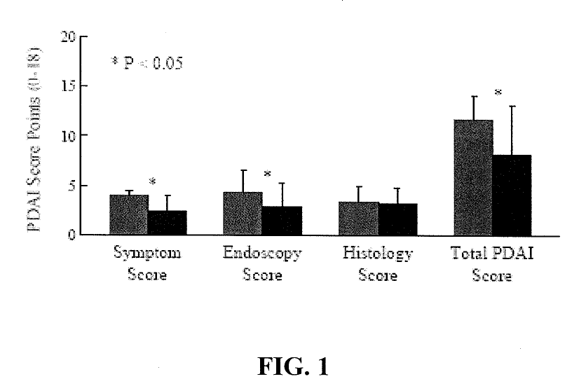

All 19 eligible patients completed the trial. Eleven patients (57.9%) had a clinical response to the therapy and 10 patients (52.6%) entered remission. For the responders, improvement of symptoms typically occurred approximately 2 weeks after initiation of the therapy. Changes in the PDAI subscores and total scores are summarized in Table 2. This data is also presented in the bar graphs depicted in FIG. 1. The P-value reported for the change is from a Wilcoxon Signed-rank test. The results showed statistically significant drops in the PDAI symptom (median drop 2.0, p=0.002), endoscopy (median drop 1.3, p=0.003), and total scores (median drop 3.9, p=0.001). The PDAI histology score had a median drop of 0 and a mean drop of 0.4, which were not statistically significant (p=0.32). Efficacy was observed in both antibiotic responsive and antibiotic refractory patients.

Dysplasia, viral inclusion bodies, or granulomas were not found in biopsy specimens from pouch endoscopy before and after the trial.

TABLE 1
Treatment Outcome (N = 19)

| | Pre treatment | Post Treatment | Reduction in Score | P value |
|---|---|---|---|---|
| PDAI Symptom Score (0-6) | 4 (4, 4) | 2 (0, 4) | −2 (−4, 0) | 0.002 |
| PDAI Endoscopy Score (0-6) | 4 (3, 5) | 3 (0, 5) | −2 (−4, 0) | 0.003 |
| PDAI Histology Score (0-6) | 3 (2, 5) | 2 (2, 3.5) | 0 (−1.5, 0.5) | 0.32 |
| Total PDAI Score (0-18) | 11 (9.5, 12.5) | 7 (3.5, 11) | −4 (−7, −0.5) | 0.001 |

*PDAI, the Pouchitis Disease Active Index; Values reported in median (interquartile range).

Although the invention has been described with reference to embodiments and examples, it should be understood that numerous and various modifications can be made without departing from the spirit of the invention. Accordingly, the invention is limited only by the following claims.

What is claimed is:

1. A method of treating one or more symptoms of pouchitis, comprising administering to a subject suffering from said symptoms adsorbent carbon.

2. The method of claim 1, wherein the adsorbent carbon is adsorbent carbon microspheres having a particle size of about 0.01 to about 2 mm.

3. The method of claim 2, further comprising identifying the subject as suffering from pouchitis.

4. The method of claim 2, wherein the adsorbent carbon microspheres are administered orally in a free flowing form.

5. The method of claim 2, wherein the symptoms are selected from one or more of diarrhea, abdominal pain, pelvic discomfort, bloody diarrhea, urgency in passing stools, or discomfort while passing stools, or abnormalities in the endoscopic appearance of the pouch, including erythematous pouch mucosa, loss of pseudocolonic vasculature or other architecture, friability of the patient's mucosa, and presence of inflammatory cells or red blood cells in the pouch lamina propria.

6. The method of claim 2, wherein the administration of the adsorbent carbon microspheres is sufficient to result in an improvement in Pouchitis Disease Activity Index.

7. The method of claim 6, wherein the improvement in the Pouchitis Disease Activity Index is at least about a 3 point reduction in the index and/or a reduction of the Pouchitis Disease Activity Index to less than 7.

8. The method of claim 2, wherein the administration of the adsorbent carbon microspheres is sufficient to result in a reduction of the Pouchitis Disease Activity Index clinical symptom score.

9. The method of claim 2, wherein the administration of the adsorbent carbon microspheres is sufficient to result in a reduction of the Pouchitis Disease Activity Index endoscopic score.

10. The method of claim 2, wherein the administration of the adsorbent carbon microspheres is sufficient to result in a reduction of the Pouchitis Disease Activity Index histology score.

11. The method of claim 2, wherein from about 1 gram to about 20 grams of the adsorbent carbon microspheres is administered daily.

12. The method of claim 2, wherein the adsorbent carbon microspheres are administered three times daily.

13. The method of claim 2, wherein the adsorbent carbon microspheres are administered four times daily.

14. The method of claim 2, wherein each dose of adsorbent carbon microspheres administered is from about 1 gram to about 6 grams.

15. The method of claim 2, wherein each dose of adsorbent carbon microspheres administered is from about 2 grams to about 3 grams.

16. The method of claim 2, wherein the adsorbent carbon microspheres have a particle size of about 0.02 to about 1 mm.

17. The method of claim 2, wherein the adsorbent carbon microspheres have a particle size of about 0.05 to about 0.8 mm.

18. The method of claim 2, wherein the adsorbent carbon microspheres have a particle size of about 0.2 to about 0.4 mm.

19. The method of claim 2, wherein the adsorbent carbon microspheres have a specific surface area of about 700 m²/g or more as determined by a BET (Brunauer-Emmett-Teller) method.

20. The method of claim 2, wherein the adsorbent carbon microspheres have a specific surface area of about 700 m²/g to about 2500 m²/g as determined by a BET (Brunauer-Emmett-Teller) method.

21. The method of claim 2, wherein the adsorbent carbon microspheres have a specific surface area of about 1500 m²/g to about 1800 m²/g as determined by a BET (Brunauer-Emmett-Teller) method.

22. The method of claim 2, wherein the volume of pores in the adsorbent carbon microspheres having a pore diameter of about 20 to about 15,000 nm is about 0.04 mL/g to about 0.10 mL/g.

23. The method of claim 2, wherein the total amount of acidic groups in the adsorbent carbon microspheres is from about 0.30 to about 1.20 meq/g.

24. The method of claim 2, wherein the total amount of basic groups in the adsorbent carbon microspheres is from about 0.20 to about 1.00 meq/g.

25. A method of treating one or more symptoms of pouchitis in a patient who continues to be symptomatic after administration of an antibiotic, the method comprising administering to the patient adsorbent carbon microspheres having a particle size of about 0.01 to about 2 mm.

26. The method of claim 25, wherein the antibiotic is selected from the group consisting of one or more of ciprofloxacin, metronidazole, and rifaximin.

27. The method of claim 25, wherein the adsorbent carbon microspheres are administered in one to four portions daily.

28. A method of treating one or more symptoms of pouchitis in a patient who is antibiotic dependent, the method comprising administering to the patient adsorbent carbon microspheres having a particle size of about 0.01 to about 2 mm.

29. The method of claim 28, wherein the antibiotic is selected from the group consisting of one or more of ciprofloxacin, metronidazole, and rifaximin.

30. The method of claim 28, wherein the adsorbent carbon microspheres are administered in one to four portions daily.

31. A method of treating one or more symptoms of pouchitis, comprising co-administering to a subject an antibiotic and adsorbent carbon microspheres having a particle size of about 0.01 to about 2 mm.

32. The method of claim 31, wherein the antibiotic is selected from the group consisting of one or more of ciprofloxacin, metronidazole, and rifaximin.

33. The method of claim 31, wherein the adsorbent carbon microspheres are administered in one to four portions daily.

34. The method of claim 31, wherein the adsorbent carbon microspheres and the antibiotic are administered simultaneously.

35. The method of claim 31, wherein the adsorbent carbon microspheres and the antibiotic are administered sequentially.

36. The method of claim 35, wherein the adsorbent carbon microspheres and the antibiotic are administered from about 20 minutes to about 1 hour apart.

37. A method of treating one or more symptoms of pouchitis, comprising co-administering to a subject a probiotic and adsorbent carbon microspheres having a particle size of about 0.01 to about 2 mm.

38. The method of claim 37, wherein the probiotic is selected from the group consisting of one or more of VSL-3 and Bio-K+.

39. The method of claim 37, wherein the adsorbent carbon microspheres and the probiotic are administered simultaneously.

40. The method of claim 37, wherein the adsorbent carbon microspheres and the probiotic are administered sequentially.

41. A method of treating one or more symptoms of pouchitis, comprising co-administering to a subject a TNF alpha inhibitor and adsorbent carbon microspheres having a particle size of about 0.01 to about 2 mm.

42. The method of claim 41, wherein the TNF alpha inhibitor is selected from the group consisting of one or more of infliximab, adalimumab, and certolizumab.

43. The method of claim 41, wherein the adsorbent carbon microspheres and the TNF alpha inhibitor are administered simultaneously.

44. The method of claim 41, wherein the adsorbent carbon microspheres and the TNF alpha inhibitor are administered sequentially.
